(19) United States
(12) Patent Application Publication
(10) Pub. No.: US 2026/0207103 A1
(43) Pub. Date: Jul. 23, 2026

(54) AUTOMATED ARTERIAL BLOOD PRESERVATION AND COLLECTION SYSTEM

(71) Applicant: MUNE CO., LTD., Seoul (KR)

(72) Inventors: Kwang Bin OH, Seoul (KR); You Hwa KIM, Hongseong-gun (KR); Jae Hak JEONG, Goyang-si (KR); Ju Han KIM, Goyang-si (KR); Dong Kyu PARK, Suwon-si (KR); Nam Yeong KIM, Busan (KR); Jae Myeong LEE, Seoul (KR)

(73) Assignee: MUNE CO., LTD., Seoul (KR)

(21) Appl. No.: 19/218,643

(22) Filed: May 27, 2025

(30) Foreign Application Priority Data
Jan. 20, 2025 (KR) 10-2025-0007958

(51) Int. Cl. A61B 5/155; A61B 5/00; A61B 5/0215; A61B 5/15; A61M 5/142; A61M 25/00

(52) U.S. Cl. CPC A61B 5/155; A61B 5/0215; A61B 5/15003; A61B 5/150992; A61B 5/6857; A61M 5/14232; A61M 2025/0003; A61M 2025/0079; A61M 2205/3317

(57) ABSTRACT

An automated arterial blood preservation and collection system, includes an arterial line in which a pressure line and a sampling line are integrated with each other so as to form a single row configuration, a pigtail opening and closing control mechanism controlling a pigtail of a transducer so as to open and close a flow of an infusion solution so that the pigtail and a peristaltic pump are operated without interfering with each other, the peristaltic pump capable of being rotated in two directions so as to suction or infuse the arterial blood inside a pump tube by pressing an outer circumferential surface of the pump tube in a longitudinal direction of the pump tube, an opening and closing control mechanism capable of being switched into any one state selected from a close contact state or a secured state, and an integrated-type pump apparatus.

### CROSS REFERENCE TO RELATED APPLICATION

The present application claims priority to Korean Patent Application No. 10-2025-0007958 (filed Jan. 20, 2025), which is hereby incorporated by reference in its entirety.

### BACKGROUND

The present disclosure relates to an automated arterial blood preservation and collection system. More particularly, the present disclosure relates to an automated arterial blood preservation and collection system configured to collect a blood sample through an arterial line without iatrogenic blood loss and configured to prevent anemia in a patient in an intensive care unit and to minimize a blood transfusion, thereby being capable of contributing to a patient blood management.

An arterial line (A-line) is a tool used for observing a hemodynamic of a patient, such as blood pressure, pulse, and so on of the patient, and for sampling arterial blood. The A-line includes a catheter inserted into the artery, a tube for connecting a saline solution bag, and a three-way valve mounted on the tube. In addition, a transducer configured to convert a pulse into an electrical signal is connected to the tube. The electrical signal generated through the transducer is transmitted to a monitor, and a doctor or a nurse may check the patient's condition through the monitor.

In a patient in an Intensive Care Unit (ICU), internal blood of the patient who uses the A-line for arterial pressure monitoring is collected through the A-line when a test such as an arterial blood gas analysis or a blood chemistry examination is performed.

Generally, when blood is collected through the A-line, initial blood of about 5 cc that is firstly collected is discarded, and then internal blood is collected. The initial blood is the blood that comes out when the blood sampling is started, and the internal blood is the blood after the initial blood is passed. The reason why the initial blood is discarded is that the initial blood is either mixed with heparin or diluted by being in contact with the saline solution even without heparin, which prevents obtaining an accurate test result value.

Therefore, in the patient in the ICU where blood sampling is performed frequently and daily, the amount of blood collected from a critical patient per day is at least about 26 ml and at most 478 ml. Since the initial blood is discarded every time the test is performed, the actual amount of blood lost from the patient is more than the amount of blood described above. According to a report, it is known that a rate of anemia occurrence of a severity or higher for each 50 cc blood collection is at least 18%, and the 97% of an inpatient is in an anemic state due to frequent blood collection.

In order to solve such a problem of blood sampling, a VAMP of Edwards Lifesciences has been introduced. The VAMP (hereinafter, the conventional product) is a blood collection apparatus in which an initial blood is not discarded and is confined to an external reservoir and then the initial blood is transfused into the body again after an internal blood is collected.

However, the conventional product has a disadvantage that a blood clot may be generated in the initial blood. The reason is that a standing blood tends to coagulate rapidly. In addition, when the initial blood flows into the reservoir, a cross-sectional area of the initial blood expands largely. Furthermore, as the initial blood accommodated in the reservoir is transfused into the body of the patient, the initial blood receives a pressure action, so that the possibility of the occurrence of the blood clot may further increase.

In addition, in the conventional product, since the reservoir is mounted between a catheter and a transducer, a damping phenomenon occurs in monitoring of an arterial blood pressure of the transducer. The damping phenomenon serves as an obstacle that disrupts a pulse signal from the arterial vessel to the transducer.

In order to solve the problems described above, Korean Patent No. 10-2507037, which has a title of “CLOSED TYPE ARTERIAL BLOOD COLLECTION APPARATUS”, previously filed and registered by the present applicant has been proposed. The closed type arterial blood collection apparatus includes: an A-line configured to connect the arterial blood vessel of a patient to a first port, the A-line being filled with a saline solution in a saline bag; a first three-way valve configured to open and close the A-line in a state of being mounted on the A-line; a blood collection unit configured to collect arterial blood through the first three-way valve when the first three-way valve closes the A-line; a second three-way valve provided between the first three-way valve and the first port, the second three-way valve being configured to open and close the A-line; a guide tube configured to connect the second three-way valve to a second port, the guide tube being filled with the saline solution in the saline bag; and a fluid conveying means configured to convey a fluid in the guide tube to the saline bag when the A-line and the guide tube are connected to each other as a result of operation of the second three-way valve. In the closed type arterial blood collection apparatus, there is an effect that a safe use is capable of being realized since there is no blood clot in the collected initial blood and there is no residual blood remaining after flushing.

However, although the previously registered disclosure of the present applicant has the effect as described above, the use of the A-line and the guide tube in two rows caused confusion to the user, and the three-way valve, the transducer, and so on are required to be manually operated by the user, so that there is a complexity of use. Therefore, the present applicant developed an apparatus capable of increasing the user convenience by solving such a problem.

### SUMMARY

Accordingly, the present disclosure has been made keeping in mind the above problems occurring in the related art, and an objective of the present disclosure is to provide an automated arterial blood preservation and collection system configured such that a sampling line and a pressure line for a blood preservation and collection have a single row configuration so as to prevent a user from being confused, the system being additionally provided with an automated apparatus so that there is no need to perform a manual operation on a three-way valve and a transducer, thereby being capable of solving a problem of complexity of use.

In order to achieve the objectives described above, according to the present disclosure, there is provided an including: an infusion solution bag in which a saline solution is accommodated and which has a lower end thereof provided with at least one port; an arterial line configured to be inserted into the arterial vessel of a patient so as to monitor a blood pressure of the patient in real time or to perform blood sampling of arterial blood of the patient, the arterial line being formed of a single row of tubes for performing a blood preservation and collection and for preventing backflow of blood; a transducer configured to convert a pulse signal of the artery of the patient delivered by using the saline solution as a medium into an electrical signal and to transmit the electrical signal to an external patient monitor; a pigtail opening and closing control mechanism configured to control a pigtail of the transducer so that the pigtail and a peristaltic pump are capable of being operated without interfering with each other; the peristaltic pump capable of being rotated in two directions such that arterial blood inside a pump tube is suctioned or infused by pressing the pump tube; an opening and closing control mechanism configured to open and close a flow of an infusion solution by controlling the peristaltic pump; and an integrated-type pump apparatus in which the transducer, the pigtail opening and closing control mechanism, the peristaltic pump, and the opening and closing control mechanism are included and mounted in one apparatus.

In the present disclosure, in a close contact state in which the opening and closing control mechanism is operated, the pigtail opening and closing control mechanism may be in an on-state such that the pigtail is opened. Furthermore, in a secured state in which the opening and closing control mechanism is not operated, the pigtail may be in an off-state such that the pigtail is closed.

In this case, in order to prevent the pigtail from interfering with the peristaltic pump during an operation of a peristaltic pump close contact apparatus, it is preferable that the pigtail opening and closing control mechanism is operated after the peristaltic pump is operated.

Meanwhile, the pigtail opening and closing control mechanism may include a pressing piece configured to adjust the flow of the infusion solution by compressing or relaxing a pigtail wing structure from both sides of the pigtail, and may include a pressing piece movement apparatus configured to move the pressing piece in the front and rear directions.

Here, an inclined part having an inclined surface formed toward a direction capable of pressing the pigtail wing structure so that the pigtail wing structure is pursed when the pressing piece is brought into contact with the pigtail wing structure and pushes the pigtail wing structure may be provided on an inner side of the pressing piece.

In addition, in order for the pigtail opening and closing control mechanism to adjust the flow of the infusion solution by compressing or relaxing the pigtail wing structure by moving the entire of transducer including the pigtail toward an inner side of the pump apparatus, a compressing piece capable of compressing the pigtail wing structure from both sides of the pigtail may be provided in the pump apparatus, and the pigtail opening and closing control mechanism may include a transducer movement apparatus configured to move the transducer in the front and rear directions.

In the present disclosure, the pigtail opening and closing control mechanism may serve as a holder on which the transducer is mounted, and may be mounted at the same height as the heart of the patient.

Meanwhile, the opening and closing control mechanism may include a pressing member configured to press and bring the pump tube into close contact with the peristaltic pump so that the peristaltic pump suctions or infuses arterial blood, and may include a pressing member movement apparatus configured to move the pressing member in the front and rear directions with respect to the peristaltic pump.

Meanwhile, the pigtail may be mounted such that a tail at a front surface portion of the pigtail is exposed outside the pump apparatus so that a user is capable of operating the pigtail by pulling the pigtail.

In the present disclosure, the arterial line may include a proximal tube which is connected to a catheter inserted into the arterial vessel of the patient and which is connected to the peristaltic pump, the pump tube wound on a rotor of the peristaltic pump in the pump apparatus; and the distal tube connected from the peristaltic pump to the port.

In addition, the automated arterial blood preservation and collection system may further include a motor current sensor configured to monitor a current in an operation of bringing the transducer into close contact with the opening and closing control mechanism or a current in an operation of securing the transducer to the opening and closing control mechanism during a normal operation of the pump apparatus, thereby being capable of checking whether the pump apparatus is operated normally.

Furthermore, the automated arterial blood preservation and collection system may further include a blockage detection sensor configured to detect a tube blockage during a normal operation of the pump apparatus in a catheterization pressure environment of the arterial line, an air injection detection sensor configured to detect and display a situation in which air bubbles are injected into the pump tube when the situation occurs during the normal operation of the pump apparatus, and a lid sensor configured to detect and display a situation in which a cover is opened or closed during the normal operation of the pump apparatus.

According to the present disclosure described above, the sampling line and the pressure line for the blood preservation and collection have the single row configuration, so that the confusion to the user may be prevented.

This configuration prevents a user error, increases the convenience of use, and realizes the safe use.

In addition, by automating the operation of the line through the opening and closing control mechanism of the pigtail and the pump tube, the complexity of use may be reduced.

### BRIEF DESCRIPTION OF THE DRAWINGS

The above and other objectives, features, and other advantages of the present disclosure will be more clearly understood from the following detailed description when taken in conjunction with the accompanying drawings, in which:

FIG. 1 is a view illustrating a configuration of an according to an embodiment of the present disclosure;

FIG. 2 and FIG. 3 are perspective views illustrating a pump apparatus according to an embodiment of the present disclosure;

FIG. 4A and FIG. 4B are perspective views illustrating a state before and after a pigtail opening and closing control mechanism according to an embodiment of the present disclosure is operated;

FIG. 5A and FIG. 5B are perspective views illustrating a state before and after the pigtail opening and closing control mechanism according to another embodiment is operated;

FIG. 6A and FIG. 6B are plan views illustrating a state before and after a peristaltic pump close contact apparatus according to an embodiment of the present disclosure is operated;

FIG. 7A and FIG. 7B are photographs showing another embodiment of the peristaltic pump close contact apparatus of the present disclosure; and

FIG. 8 to FIG. 10 are views illustrating a process of performing blood sampling by using the automated arterial blood preservation and collection system illustrated in FIG. 1.

### DETAILED DESCRIPTION

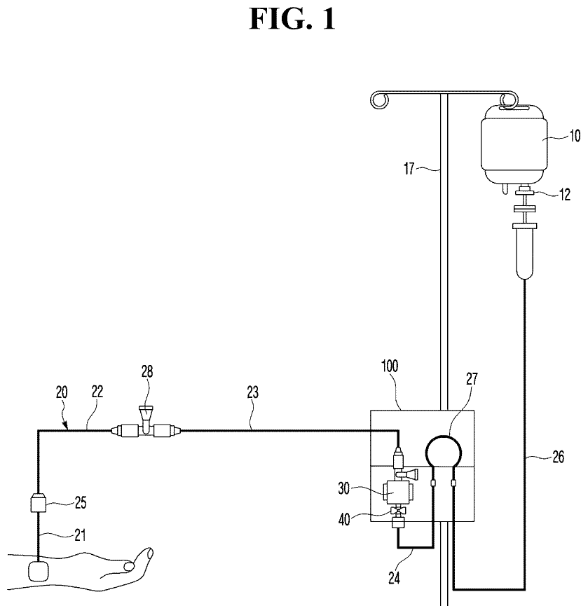

FIG. 1 is a view illustrating a configuration of an according to an embodiment of the present disclosure.

As illustrated in the drawing, the automatic arterial blood preservation and collection system according to the present embodiment includes an infusion solution bag 10, an arterial line 20, a transducer 30, a pigtail opening and closing control mechanism, a peristaltic pump, an opening and closing control mechanism, and an integrated-type pump apparatus 100.

The infusion solution bag 10 is a plastic bag in which a saline solution is accommodated, and a lower end of the infusion solution bag 10 is provided with at least one port 12. An inner portion of the infusion solution bag 10 is provided with a pressure bag which maintains a pressure of about 300 mmHg and which is configured to apply a constant pressure to the infusion solution bag 10.

As the pressure bag maintains the pressure of 300 mmHg, the pressure bag supports a supply of a flush solution and supports a prevention of blood backflow of the arterial line 20, and the saline solution in the infusion solution bag 10 may include heparin. Similar to a conventional saline solution bag, the infusion solution bag 10 is mounted on an upper end portion of a pole 17.

The arterial line 20 is provided for monitoring a blood pressure of a patient in real time or for collecting an arterial blood, and provides a passage connecting the port 12 and the arterial vessel of the patient to each other.

The arterial line 20 in the present disclosure is formed of a single row of tubes which include a pressure line configured to deliver a pressure and a flow rate of the pressure bag to the patient so that blood does not backflow and which include a sampling line configured to perform a preservation and collection of blood.

That is, the present disclosure provides the automated arterial blood preservation and collection system capable of draining an initial blood in the arterial vessel diluted by a normal saline solution when the arterial blood is collected, then collecting a required amount of internal blood, and then retransfusing the initial blood back into the body. Furthermore, the automated arterial blood preservation and collection system has a configuration in which the sampling line for performing blood sampling is integrated with the pressure line so as to form a single-row configuration.

The arterial line 20 in which the sampling line is integrated with the pressure line is connected from the arterial vessel of the patient to the port 12 as a single row of tubes. Furthermore, the arterial line 20 is configured such that a pigtail and the peristaltic pump of the transducer 30 are capable of being operated without interfering with each other during performing blood sampling while the arterial line 20 performs a function of the pressure line configured to deliver the pressure and the flow rate of the pressure bag so that the blood does not backflow in the arterial vessel of the patient.

To this end, in the present disclosure, the pigtail opening and closing control mechanism and the opening and closing control mechanism are provided, and will be described later.

Here, the arterial line 20 may include proximal tubes 22, 23, and 24 (a first proximal tube 22, a second proximal tube 23, and a third proximal tube 24) connected to a catheter 21 inserted into the blood vessel and connected to the peristaltic pump, a pump tube 27 wound on a rotor of the peristaltic pump in the pump apparatus 100, and a distal tube 26 connected from the peristaltic pump to the port 12.

The proximal tubes 22, 23, and 24, the pump tube 27, and the distal tube 26 are connected to each other so as to form a single passage, the arterial line 20 formed of the proximal tubes 22, 23, and 24, the pump tube 27, and the distal tube 26 forms the pressure line configured to deliver the pressure and the flow rate of the pressure bag to the patient, and a partial region of the arterial line 20 passing through the transducer 30 of the pump apparatus 100 may form the sampling line configured to perform blood sampling.

That is, the sampling line may be formed by a length up to a length of the proximal tubes 22, 23, and 24, and may help with a patient blood management by minimizing blood transfusion to the patient since the initial blood is retransfused into the body again after the required amount of internal blood is collected.

The proximal tubes 22, 23, and 24 are in communication with the catheter 21 through a three-way valve 25 and are connected to the peristaltic pump. Furthermore, the proximal tubes 22, 23, and 24 may include the first proximal tube 22 connected from the three-way valve 25 to a port 28 capable of being connected to another apparatus, the second proximal tube 23 connected from the port 28 to the transducer 30, and the third proximal tube 24 connected from the transducer 30 to the peristaltic pump. The first proximal tube 22 and the second proximal tube 23 may be formed as one tube, but there is no limitation in the present disclosure.

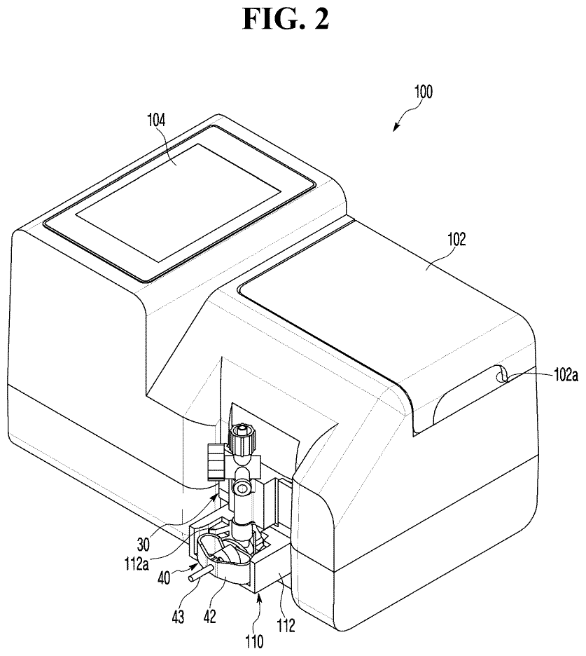

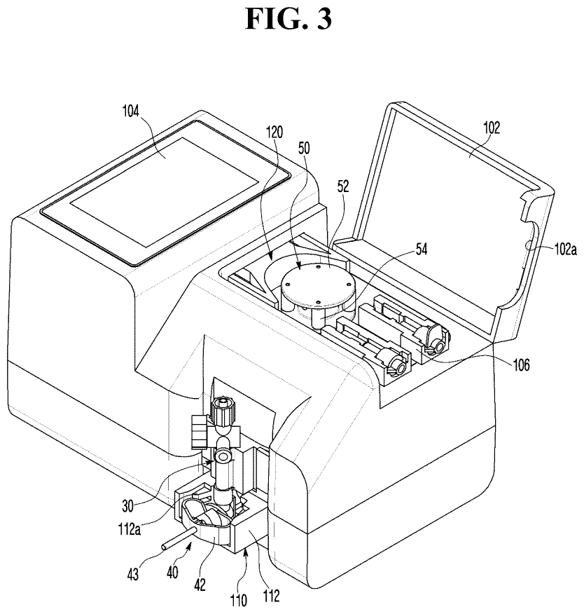

FIG. 2 and FIG. 3 are perspective views illustrating a pump apparatus according to an embodiment of the present disclosure, and the pump apparatus 100 has a configuration in which the transducer 30, a pigtail opening and closing control mechanism 110, a peristaltic pump 50, and an opening and closing control mechanism 120 are mounted in a single apparatus.

The pump apparatus 100 is formed in a substantially hexahedral shape. Furthermore, an upper surface of the pump apparatus 100 is provided with a touch panel 104 having a display function in which a user operates the apparatus by a touch operation and a state of the apparatus is informed, and is provided with a cover 102 mounted on an upper portion of the peristaltic pump 50.

The touch panel 104 is configured to control the operation of the peristaltic pump 50 more precisely, and is also configured to display an operation situation of the pump apparatus 100 in real time. A doctor or a nurse may visually check the operation situation of the pump apparatus 100 through the touch panel 104.

The cover 102 is capable of being opened and closed around a hinge at a first side of the cover 102, and a tube mounting hole 102a for mounting a tube in the peristaltic pump 50 is formed in the cover 102.

The transducer 30 and a pigtail 40 are mounted on a front surface of the pump apparatus 100 while the transducer 30 and the pigtail 40 are in a state in which the transducer 30 and the pigtail 40 are exposed to the outside. The pigtail 40 is mounted such that a tail 43 of a front surface portion of the pigtail 40 is exposed to the outside of the pump apparatus 100 so that the user is capable of operating the pigtail 40 by pulling the pigtail 40.

The transducer 30 is configured to convert a pulse signal of the artery of the patient transmitted by using a saline solution as a medium into an electrical signal and to transmit the electrical signal to an external patient monitor, and a structure and a function of the transducer 30 are general, so that a description of the structure and the function of the transducer 30 will be omitted.

In the present disclosure, in order to control the pigtail 40 of the transducer 30 such that the pigtail 40 and the peristaltic pump 50 are capable of being operated without interfering with each other, the pigtail opening and closing mechanism 110 configured to open and close a flow of the infusion solution from the infusion solution bag 10 to the patient is provided, and the opening and closing control mechanism 120 configured to select an operation state of the peristaltic pump 50 is provided.

The pigtail opening and closing control mechanism 110 is configured to control opening and closing of the pigtail 40 so that the flow of the infusion solution is adjusted by compressing or relaxing a pigtail wing structure 42 from both sides of the pigtail 40. In an embodiment illustrated in FIG. 2 and FIG. 3, the embodiment of the present disclosure may include a pressing piece 112 for compressing and relaxing the pigtail wing structure 42 from the both sides of the pigtail 40, and may include a pressing piece movement apparatus for moving the pressing piece 112 in the front and rear directions.

An inclined part 112a configured to be in contact with the pigtail wing structure 42 is formed on an inner side surface of the pressing piece 112. The inclined part 112a is formed such that the inclined part 112a has an inclined surface toward a direction capable of pressing the pigtail wing structure 42 so that the pigtail wing structure 42 is pursed when the pressing piece 112 is in contact with the pigtail wing structure 42 and pushes the pigtail wing structure 42, so that the pigtail 40 is capable of being opened 40 as the pressing piece movement apparatus is operated.

In the pressing piece movement apparatus, a known technology may be applied when the pressing piece movement apparatus has a structure capable of moving the pressing piece 112 in the front and rear directions. For example, the pressing piece movement apparatus capable of performing a linear movement precisely may be provided by coupling a motor and a linear guide to each other, and various technologies may be applied thereto, so that a description of the configuration of the pressing piece movement apparatus is omitted in the present disclosure.

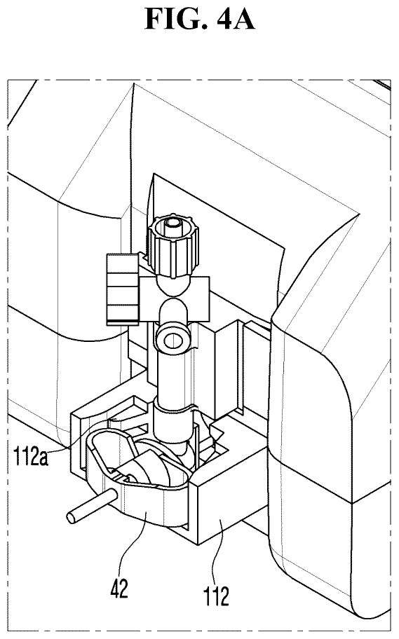

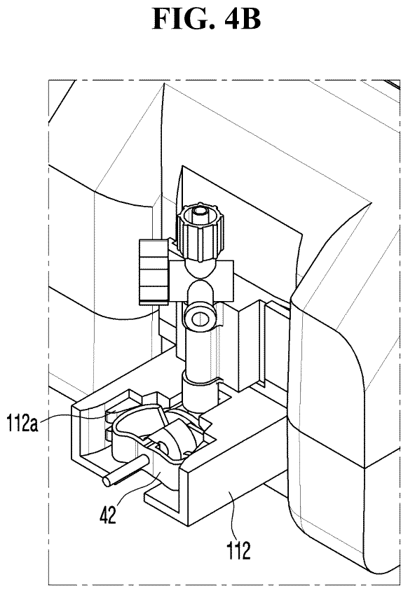

FIG. 4A and FIG. 4B are perspective views illustrating a state before and after the pigtail opening and closing control mechanism according to an embodiment of the present disclosure is operated, FIG. 4A is a perspective view illustrating a state in which the pigtail 40 is closed, and FIG. 4B is a perspective view illustrating a state in which the pigtail 40 is opened by pressing the pigtail wing structure 42 from the both sides of the pigtail wing structure 42 as the pressing piece movement apparatus is operated and the pressing piece 112 is moved in the front direction.

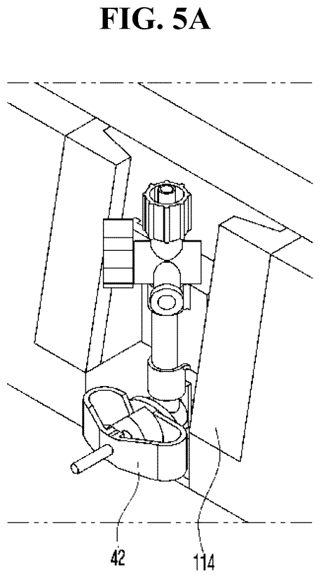

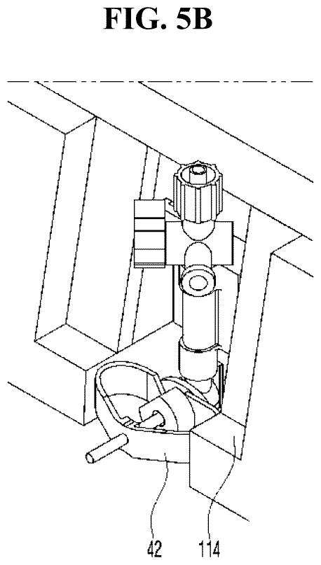

Meanwhile, FIG. 5A and FIG. 5B are views illustrating another embodiment of the pigtail opening and closing control mechanism of the present disclosure, and the pigtail opening and closing control mechanism may be configured such that the entire of the transducer 30 including the pigtail 40 is capable of controlling the flow of the infusion solution by compressing or relaxing the pigtail wing structure 42 as the transducer 30 is moved toward an inner side of the pump apparatus 100.

In this situation, a compressing piece 114 capable of compressing the pigtail wing structure 42 from the both side of the pigtail wing structure 42 may be provided in the pump apparatus 100, and a transducer movement apparatus for moving the transducer 30 in the front and rear directions may be included.

A known technology is capable of being applied to the transducer movement apparatus, and there is no limitation in the present disclosure.

In another embodiment of such a pigtail opening and closing control mechanism, the compressing piece 114 is mounted such that a height of the compressing piece 114 is equal to a height of the pigtail wing structure 42, so that the compressing piece 114 is brought into contact with the pigtail wing structure 42 and the pigtail wing structure 42 is compressed as the transducer movement apparatus moves the transducer 30 in the rear direction.

In the present disclosure, the pigtail opening and closing control mechanism serves as a holder on which the transducer 30 is mounted, and is mounted at the same height as the heart of the patient.

Meanwhile, the peristaltic pump 50 for suctioning the initial blood and then infusing the initial blood again after the blood sampling is performed is mounted in the pump apparatus 100.

The peristaltic pump 50 is mounted such that the peristaltic pump 50 is capable of being rotated in two directions so that the peristaltic pump 50 presses an outer circumferential surface of the pump tube 27 in a longitudinal direction of the pump tube 27 so as to suction or infuse the arterial blood inside of the tube, and includes a rotor 52 configured to perform a bi-directional rotation, a motor (not illustrated) configured to rotate the rotor 52, and a plurality of pressing rolls 54 fixed to the rotor 52 in a circumferential direction of the rotor 52.

The pump tube 27 is mounted on the pressing rolls 54 such that the pump tube 27 surrounds the pressing rolls 54 while the pump tube 27 is in a state in which the pump tube 27 is fitted to a tube supporting part 106 of the pump apparatus 100, and the opening and closing control mechanism 120 is mounted such that the opening and closing control mechanism 120 is capable of pressing the pump tube 27 surrounding the pressing rolls 54.

Meanwhile, the opening and closing control mechanism 120 includes a pressing member 122 configured to press and bring the pump tube 27 into close contact the peristaltic pump 50 so that the peristaltic pump 50 suctions or infuses the arterial blood, and includes a pressing member movement apparatus configured to move the pressing member 122 in front and rear direction with respect to the peristaltic pump 50.

The pressing member 122 has an arc shape corresponding to the outer circumferential surface of the rotor 52 of the peristaltic pump 50, and may be formed in an angle range including at least 120 degrees.

The pump tube 27 may be pressed as the pressing member 122 moves toward the peristaltic pump 50 and presses the pump tube 27 surrounding the pressing rolls 54.

The opening and closing control mechanism 120 of the present disclosure is configured to be in any one state selected from a close contact state in which the peristaltic pump 50 presses the outer circumferential surface of the pump tube 27 so that the peristaltic pump 50 suctions or infuses the arterial blood and a secured state in which pressing on the pump tube 27 is released and the pressure of the infusion solution bag 10 is delivered to the patient, and these states may be divided into three states in the present disclosure.

(1) Non-secured state: this is a state in which the user does not mount the pump tube 27 or the pump tube 27 is mounted incorrectly.

(2) Secured state: this is a state in which the user correctly mounts the pump tube 27 and the infusion solution inside the pump tube 27 flows properly.

(3) Close contact state: this is a state in which the pump tube 27 is in close contact with the rotor 52 of the peristaltic pump 50 so that the flow of the infusion solution inside the pump tube 27 is stopped and the infusion solution flows only by the peristaltic pump.

In these three states, the secured state is a state in which the opening and closing control mechanism 120 is not operated and the pressing on the pump tube 27 is not performed, and the pressure of the infusion solution bag 10 is delivered to the patient.

The close contact state is a state in which the opening and closing control mechanism 120 is operated. That is, when the opening and closing control mechanism 120 is operated, the pump tube 27 is pressed by the pressing rolls 54, and the pressing rolls 54 transfer the fluid inside the pump tube 27 as the pressing rolls 54 push the pump tube 27 and are rotated.

In the present disclosure, in the close contact state in which the opening and closing control mechanism 120 is operated, the pigtail opening and closing control mechanism 110 is in an on-state such that the pigtail 40 is opened. Furthermore, in the secured state in which the opening and closing control mechanism 120 is not operated, the pigtail opening and closing control mechanism 110 is in an off-state such that the pigtail 40 is closed.

In this situation, in order for the pigtail 40 to not interfere with the peristaltic pump 50 when the opening and closing control mechanism 120 is operated, it is preferable that the pigtail opening and closing control mechanism 110 is operated after the peristaltic pump 50 is operated, and it is preferable that the operation of the pigtail opening and closing control mechanism 110 is operated within 0.1 seconds to 1 second after the peristaltic pump 50 is operated.

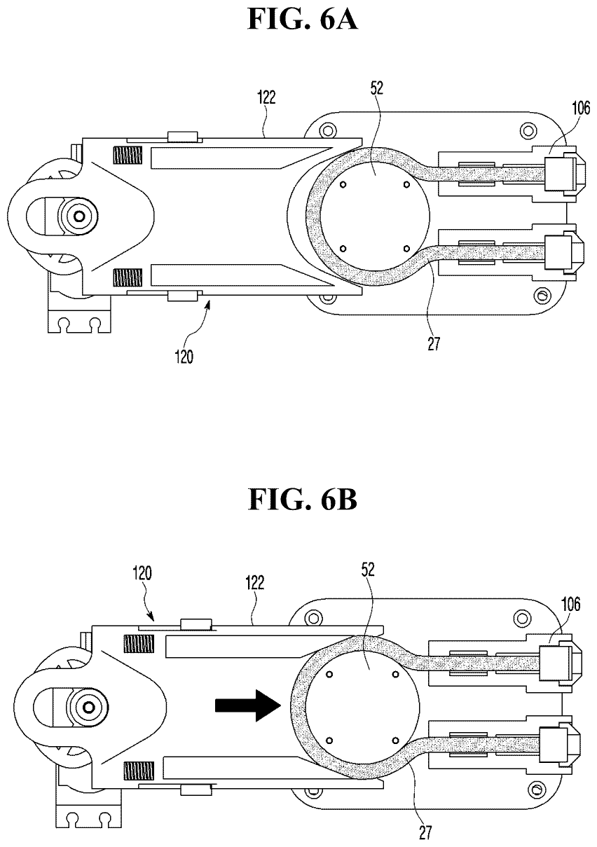

FIGS. 6A and 6B are plan views illustrating a state before and after a peristaltic pump close-contact apparatus according to an embodiment of the present disclosure is operated, FIG. 6A illustrates the secured state in which the pump tube 27 the mounted on the rotor 52. In the secured state, since the opening and closing control mechanism 120 is not operated, the pressing on the pump tube 27 is not performed, so that the pressure of the infusion solution bag 10 is delivered to the patient.

FIG. 6B illustrates the close contact state in which the opening and closing control mechanism 120 is operated and the pump tube 27 is in close contact with the peristaltic pump 50. In the close contact state, when the opening and closing control mechanism 120 is operated, the pump tube 27 is pressed by the pressing rolls 54, and the pressing rolls 54 transfer the fluid inside the pump tube 27 as the pressing rolls 54 push the pump tube 27 and are rotated.

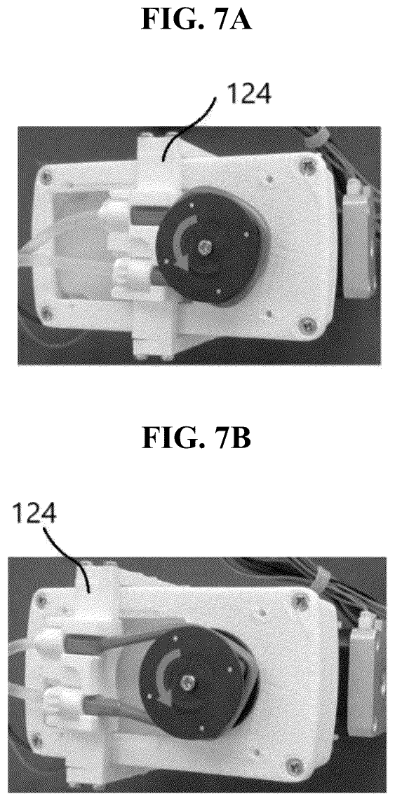

FIG. 7A and FIG. 7B are photographs showing another embodiment of the peristaltic pump close contact apparatus of the present disclosure.

The opening and closing control mechanism 120 may include a connector 124 configured to fix both ends of the pump tube 27 wound on the rotor 52 of the peristaltic pump 50, and may include a connector movement apparatus configured to bring the pump tube 27 into close contact with the rotor 52 of the peristaltic pump 50 by pulling the connector 124 in a direction away from the peristaltic pump 50.

FIG. 7A illustrates the secured state in which the pump tube 27 is mounted on the rotor 52 of the peristaltic pump 50. In the secured state, the connector movement apparatus is not operated, and the pressing on the pump tube 27 is not performed, so that the pressure of the infusion solution bag 10 is delivered to the patient.

In FIG. 7B, as the connector movement apparatus pulls the connector 124 in the direction away from the peristaltic pump 50 so that the pump tube 27 is brought into close contact with the rotor 52 of the peristaltic pump 50, the pump tube 27 is pressed by the pressing rolls 54, and the pressing rolls 54 transfer the fluid inside the pump tube 27 by pressing the pump tube 27 and being rotated.

Meanwhile, the pump apparatus 100 of the present disclosure may include a motor current sensor (not illustrated) configured to monitor a current in an operation of bringing the transducer 30 into close contact with the opening and closing control mechanism 120 or in an operation of securing the transducer 30 to the opening and closing control mechanism 120 during the normal operation of the pump apparatus 100, thereby being capable of checking whether the pump apparatus 100 is normally operated.

In addition, the pump apparatus 100 may include a blockage detection sensor (not illustrated) configured to detect a tube blockage during the normal operation of the pump apparatus 100 in a catheterization pressure environment of the arterial line 20, an air injection detection sensor (not illustrated) configured to detect and display a situation in which air bubbles are injected into the tube when the situation occurs during the normal operation of the pump apparatus 100, and a lid sensor configured to detect and display a situation in which the cover 102 is opened or closed during the normal operation of the pump apparatus 100.

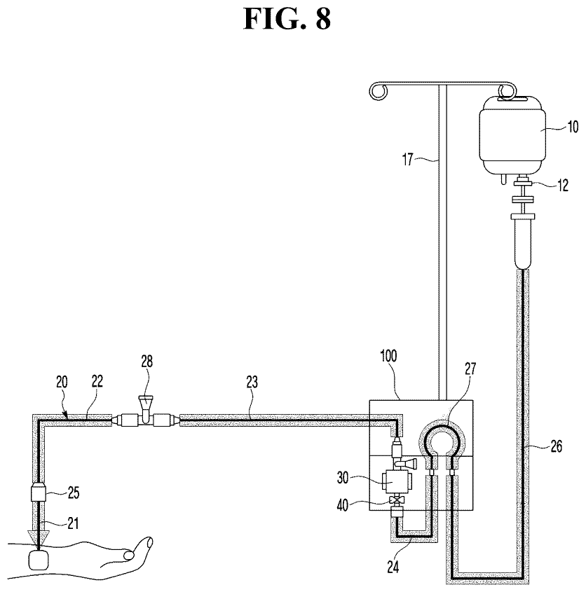

FIG. 8 to FIG. 10 are views illustrating a process of performing blood sampling by using the automated arterial blood preservation and collection system illustrated in FIG. 1.

FIG. 8 illustrates a state in which the catheter 21, the proximal tubes 22, 23 and 24, the pump tube 27, and the distal tube 26 are connected to each other by opening the arterial line 20, and the saline solution including heparin is filled inside the arterial line 20.

The pressure inside the arterial line 20 and the pressure of the arterial vessel are adjusted to an atmospheric pressure through a separate zeroing process, a pulse of the heart is delivered to the transducer 30 by the arterial blood and the saline solution inside the proximal tube 23 as the medium, and the transducer 30 converts a pulse signal into an electrical signal and transmits the electrical signal to the patient monitor (not illustrated).

In this state, in order to perform blood collecting, the opening and closing control mechanism 120 is operated such that the state of the peristaltic pump 50 is switched from the secured state to the close contact state, and the pigtail 40 is opened by operating the pigtail opening and closing control mechanism 110.

After that, a regurgitation suction is performed so as to suction 7 cc of blood at a rate of 1 cc/s.

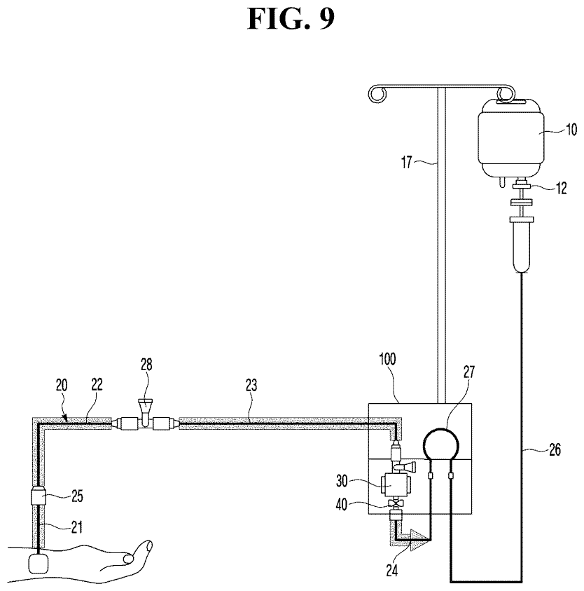

The initial blood is suctioned in the direction of the arrow illustrated in FIG. 9, so that the initial blood is induced inside the proximal tubes 22, 23, and 24. The portion of the arterial line 20 through which the blood sampling of the initial blood is induced becomes the sampling line, and the sampling line may extend from the catheter 21 to a partial region passing through the transducer 30 of the pump apparatus 100.

After the suction is completed and the system is ready for performing blood sampling, a blood collection syringe is inserted into a blood collection port and collects the internal blood.

When the blood sampling of the internal blood is completed, the three-way valve 25 is opened again, and the peristaltic pump 50 is rotated in the reverse direction.

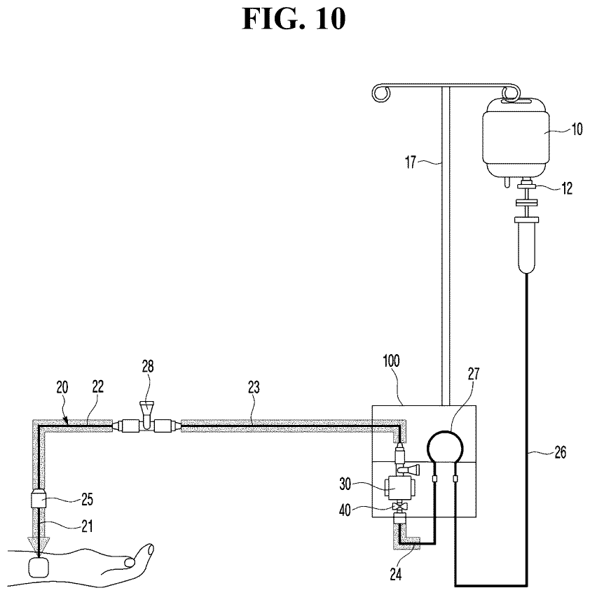

As the peristaltic pump is rotated in the reverse direction, the internal blood and the initial blood that were suctioned into the tube of the sampling line are moved in the direction of the arrow in FIG. 10, and the internal blood and the initial blood are reinfused into the arterial blood vessel.

After the reinfusion, the state of the peristaltic pump 50 is switched from the close contact state to the secured state, the pigtail 40 is closed by operating the pigtail opening and closing control mechanism 110, and the system is in a standby state.

In the pump apparatus 100 of the present disclosure, the pump apparatus 100 may be configured such that the pump apparatus 100 generates alarm sounds that inform the completion of suction and the reinfusion, and a predetermined interval (for example, 20 seconds) may be set between the alarm sounds informed by the pump apparatus 100, so that blood sampling is capable of being performed between the suction completion alarm sound and the reinfusion alarm sound.

In this situation, the forward direction rotation and the reverse direction rotation of the peristaltic pump 50 are set to be automatically operated at the same interval as the alarm sounds. Therefore, when the blood sampling is completed, the blood and the saline solution are reinfused into the patient.

Therefore, in the automated arterial blood preservation and collection system of the present disclosure, there is an effect that the suction amount and the infusion amount of the blood are reduced compared to that of a two-column structure in which the sampling line is separately provided from the pressure line.

In the two-column structure, the suction amount is 7.5 cc and the infusion amount is 19.5 cc, whereas the suction amount and the infusion amount in an embodiment of the present disclosure may be 5.5 cc and 12.5 cc by configuring the sampling line and the pressure line as the single row configuration.

As such, by reducing the suction amount for the blood preservation and collection, the present disclosure have effects that the blood sampling amount is capable of being reduced and the patient blood management is capable of being assisted by minimizing the blood transfusion to the patient.

Although the exemplary embodiments of the present disclosure have been described above, the scope of the present disclosure is not limited thereto. The scope of the rights of the present disclosure extends to those that are substantially equivalent to the embodiments of the present disclosure, and various modifications may be made by those skilled in the art to which the present disclosure pertains without departing from the spirit of the present disclosure.

1. An automated arterial blood preservation and collection system comprising:
an infusion solution bag in which a saline solution is accommodated and which has a lower end thereof provided with at least one port;
an arterial line configured to be inserted into the arterial vessel of a patient so as to monitor a blood pressure of the patient in real time or to perform blood sampling of arterial blood of the patient, the arterial line being formed of a single row of tubes for performing a blood preservation and collection and for preventing backflow of blood;
a transducer configured to convert a pulse signal of the artery of the patient delivered by using the saline solution as a medium into an electrical signal and to transmit the electrical signal to an external patient monitor;
a pigtail opening and closing control mechanism configured to control a pigtail of the transducer so that the pigtail and a peristaltic pump are capable of being operated without interfering with each other;
the peristaltic pump capable of being rotated in two directions such that arterial blood inside a pump tube is suctioned or infused by pressing the pump tube;
an opening and closing control mechanism configured to open and close a flow of an infusion solution by controlling the peristaltic pump; and
an integrated-type pump apparatus in which the transducer, the pigtail opening and closing control mechanism, the peristaltic pump, and the opening and closing control mechanism are included and mounted in one apparatus.

2. The automated arterial blood preservation and collection system of claim 1, wherein, in a close contact state in which the opening and closing control mechanism is operated, the pigtail opening and closing control mechanism is in an on-state such that the pigtail is opened, and
in a secured state in which the opening and closing control mechanism is not operated, the pigtail is in an off-state such that the pigtail is closed.

3. The automated arterial blood preservation and collection system of claim 2, the pigtail opening and closing control mechanism is operated after the peristaltic pump is operated, thereby preventing the pigtail to interfere with the peristaltic pump when the opening and closing control mechanism is operated.

4. The automated arterial blood preservation and collection system of claim 1, wherein the pigtail opening and closing control mechanism comprises:
a pressing piece configured to adjust the flow of the infusion solution by compressing or relaxing a pigtail wing structure from both sides of the pigtail; and
a pressing piece movement apparatus configured to move the pressing piece.

5. The automated arterial blood preservation and collection system of claim 1, wherein, in order for the pigtail opening and closing control mechanism to adjust the flow of the infusion solution by compressing or relaxing a pigtail wing structure by moving the entire of transducer including the pigtail toward an inner side of the pump apparatus, a compressing piece capable of compressing the pigtail wing structure from both sides of the pigtail is provided in the pump apparatus, and the pigtail opening and closing control mechanism comprises a transducer movement apparatus configured to move the transducer in the front and rear directions.

6. The automated arterial blood preservation and collection system of claim 1, wherein the pigtail opening and closing control mechanism serves as a holder on which the transducer is mounted, and is mounted at the same height as the heart of the patient.

7. The automated arterial blood preservation and collection system of claim 1, wherein the opening and closing control mechanism comprises:
a pressing member configured to press and bring the pump tube into close contact with the peristaltic pump so that the peristaltic pump suctions or infuses arterial blood; and
a pressing member movement apparatus configured to move the pressing member in the front and rear directions with respect to the peristaltic pump.

8. The automated arterial blood preservation and collection system of claim 1, wherein the pigtail is mounted such that a tail at a front surface portion of the pigtail is exposed outside the pump apparatus so that a user is capable of operating the pigtail by pulling the pigtail.

9. The automated arterial blood preservation and collection system of claim 1, wherein the arterial line comprises:
a proximal tube which is connected to a catheter inserted into the arterial vessel of the patient and which is connected to the peristaltic pump;
the pump tube wound on a rotor of the peristaltic pump in the pump apparatus; and
a distal tube connected from the peristaltic pump to the port.

10. The automated arterial blood preservation and collection system of claim 1, further comprising a motor current sensor configured to monitor a current in an operation of bringing the transducer into close contact with the opening and closing control mechanism or a current in an operation of securing the transducer to the opening and closing control mechanism during a normal operation of the pump apparatus, thereby being capable of checking whether the pump apparatus is operated normally.

11. The automated arterial blood preservation and collection system of claim 1, further comprising:
a blockage detection sensor configured to detect a tube blockage during a normal operation of the pump apparatus in a catheterization pressure environment of the arterial line;
an air injection detection sensor configured to detect and display a situation in which air bubbles are injected into the pump tube when the situation occurs during the normal operation of the pump apparatus; and
a lid sensor configured to detect and display a situation in which a cover is opened or closed during the normal operation of the pump apparatus.
